(19) United States
(12) Patent Application Publication
(10) Pub. No.: US 2011/0271965 A1
(43) Pub. Date: Nov. 10, 2011

(54) Multi-Material Hearing Protection Custom Earplug

(75) Inventors: John W. Parkins, Ithaca, NY (US); Mark DeWilde, Ithaca, NY (US)

(73) Assignee: RED TAIL HAWK CORPORATION, Ithaca, NY (US)

(21) Appl. No.: 13/103,417

(22) Filed: May 9, 2011

Related U.S. Application Data
(60) Provisional application No. 61/332,929, filed on May 10, 2010.

(51) Int. Cl. A61F 11/08; G06F 17/50

(52) U.S. Cl. 128/867; 128/864; 700/98

(57) ABSTRACT

An earplug formed of a plurality of materials having different hardnesses by use of a multi-material rapid prototyping (RP) system.

### REFERENCE TO RELATED APPLICATIONS

This application claims one or more inventions which were disclosed in Provisional Application No. 61/332,929, filed May 10, 2010, entitled “Multi-material hearing protection custom earplug”. The benefit under 35 USC §119(e) of the United States provisional application is hereby claimed, and the aforementioned application is hereby incorporated herein by reference.

### ACKNOWLEDGMENT OF GOVERNMENT SUPPORT

This invention was made with Government support under SBIR contract N68335-10-C-0329, awarded by the US Navy. The government has certain rights in the invention.

### BACKGROUND OF THE INVENTION

1. Field of the Invention

The invention pertains to the field of earplugs. More particularly, the invention pertains to earplugs made of a number of materials having different hardnesses, and a method of fabrication of such earplugs.

2. Description of Related Art

This field is similar to the manufacture of custom fitted hearing aids, particularly to devices that fit deeply into the ear canal. Typically, an impression of the ear canal and concha are made by injecting a silicone material into the ear canal, allowing it to harden, and then withdrawing it from the ear to produce an accurate representation of the ear canal shape. The impression may be mechanically altered and used to produce a mould of the desired device, after which the device is cast into the mould and then finished. In a more modern approach, the impression is optically or mechanically scanned and the digital representation further processed using a computer program to create the final device shape. To convert the digital model to a final device or into a mould to use to cast the final device, a single material rapid prototyping (RP) process is employed (see Parsi et al, US2010/0026775).

Deep insertion ear plugs today are made either of relatively hard materials that can be produced by rapid prototyping methods, or of silicone elastomers which must be cast into moulds that are often produced by the RP methods. The materials used in common visco-elastic foam ear plugs attenuate sound efficiently, but are exceedingly difficult to insert deeply into the ear canal where they need to be placed in order to perform.

There is advantage in having the part of the plug in the outer portion of the ear canal be made of hard materials to contain and protect electronics assemblies, and the part of the plug in the interior portion of the ear canal made of softer material to allow flexing and bending while being inserted, and to reduce movement of the plug when the canal shape changes due to jaw movement. Traditional manufacturing methods would require the plug to be made in 2 (or more) parts, some hard and the others cast in soft material. The parts would then be glued together or mechanically interconnected. This assembly method introduces joints which can collect contamination or can fail. It also requires additional manufacturing steps, and limits the mechanical configurations possible.

When using soft materials formed in a mould, a limitation is encountered on the geometry of interior cavities and openings due to the process. There are often one or more air or sound passages that must be incorporated into the device for tailored acoustic response. There can be an advantage to having these passages possess complex shapes and have varying dimensions. In a casting process, a core that has the desired shape must be precisely placed in the mould and the part cast around it. After hardening, the core must be removed mechanically or by dissolving out. Both of these methods place restrictions on the sizes and geometries permitted and also on the sizes and numbers of passages possible.

Zwislocki (U.S. Pat. No. 2,803,247) describes an elastomer shell filled with a sound absorbing viscous fluid or soft wax. The method he describes requires multiple manufacturing steps and requires a method to introduce the material, which leads to potential leakage.

In Garcia (U.S. Pat. No. 5,742,692) a device is illustrated with a hard body covered with a softer material and fitted with a soft tip. Multiple mechanical joints and a relatively large number of parts make the design expensive and impractical.

Touson (U.S. Pat. No. 2,934,160) shows an ear plug consisting of a thin flexible shell filled with a liquid, and incorporating a channel to allow the insertion of a sound tube. The channel shape shown has a spherical expansion in the center which allows for a sound horn on the end of the sound tube. The device must be manufactured as a shell, then filled with the fluid and sealed, and then the sound tube installed via stretching the walls of the shell channel. The concept suffers from difficulty of installing the sound tube, and the need for multiple manufacturing steps.

There are situations where the embedding of hard materials within a matrix of soft material has advantages. Mendelson (U.S. Pat. No. 3,131,241) describes an ear plug made by a combination of casting an air filled elastomeric hollow shell and gluing in a stiff tube to provide strength while inserting the ear plug. The device is hollow and is sealed so that air pressure provides support of the outer elastomeric walls. A similar structure is described in Mills (U.S. Pat. No. 3,736,929) wherein an elastomeric shell is filled with sound absorbing filler and fitted with a central tube to act as a stiffener.

Active Hearing devices contain sound transducer elements as well as electronics. These elements benefit from being isolated from surrounding sources of vibration. In conventional manufacture, the addition of tiny elastomeric components or layers of waxy sound absorbing material to isolate these elements is both difficult and impractical from a manufacturing standpoint.

The rapid prototyping process may be based on several technologies. The rapid prototyping methods until very recently have been capable only building the object up from a single material which is solidified from a solid powder by a laser sintering process (see Jandeska et al, U.S. Pat. No. 7,141,207), or from a liquid via a photo-polymerization process. Solid materials are typically blown onto the surface from a bulk reservoir, and then fused onto the previous later via application of laser heating in specific areas. The liquid materials have been supplied from a bulk bath where the object is built up in layers by solidifying the surface of the liquid and then lowering the solidified layer deeper into the bulk tank (see Wahlstrom et al U.S. Pat. No. 7,585,450; Walstrom, U.S. Pat. No. 7,690,909; Reynolds et al, U.S. Pat. No. 7,621,733; Henningsen, U.S. Pat. No. 7,128,866), by depositing a layer on a surface and polymerizing the desired portions, removing the uncured material and then adding the next layer (see Sperry et al, U.S. Pat. No. 7,614,866; Huang et al, U.S. Pat. No. 7,158,849), or by ink-jet deposition and subsequent optical polymerization.

The newest methods employ an ink-jet printing type of process, wherein both a support material and a modeling material are applied in layers and photo-polymerized (see Vanmaele et al, US2010/0007692). When complete, the support material is washed away leaving the finished model.

Earlier ink jet technologies permitted materials to be changed after a group of layers had been set down, but did not allow materials to be mixed in different regions of a single layer. There are now versions of this equipment that support the application of multiple materials on each layer, permitting the creation of composites and intermixed materials (see Eshed et al, US2009/0210084; Kritchman, US20090148621 and US2009/0145357). Both soft (elastomeric) and hard materials are available and may be freely intermixed. Bonding between dissimilar materials is excellent; no glue is required.

One of the most significant barriers to the use of multi-material RP processes has been the availability of materials with the characteristics needed to be compatible with the RP machine, to also provide the mechanical properties desired, and to have biocompatibility—the ability to remain in contact with sensitive skin for long periods without allergic reactions or sensitivity. In this latter regard, silicones have proven to be excellent materials to use, but are not compatible with the RP machines.

Sound transducers and other external access ports must have seals to the shell of the device. Typically when using hard materials to surround and protect the active components in a device, tiny o-rings or other types of seals must be installed in the housing as part of final assembly. The additional parts and difficulty of assembling these tiny components adds significantly to the cost of the assembly.

### SUMMARY OF THE INVENTION

The earplug of the invention is formed of a plurality of materials having different hardnesses by use of a multi-material rapid prototyping (RP) system.

### BRIEF DESCRIPTION OF THE DRAWING

FIGS. 1a and 1b show two views of an earplug made with three different material sections.

FIG. 2 shows a photograph of an earplug made of three separate material sections using ink-jet printing technology.

FIG. 3 is a flowchart of a method of fabricating the earplug.

FIG. 4 is a sectional drawing of a representative earplug.

FIG. 5 is a sectional drawing of an earplug with electronic components housed in the earplug.

### DETAILED DESCRIPTION OF THE INVENTION

The combination of biocompatible coatings and multi-material ink-jet rapid prototyping creates a technology that can be effectively applied to solve a number of problems encountered in the design and manufacture of custom hearing protection and enhancement devices. A number of capabilities and properties of the process when applied in unique ways can reduce costs and optimize designs for manufacturability and performance.

### The Earplug

The earplug of the invention is formed of a plurality of materials having different hardnesses by use of a multi-material rapid prototyping (RP) system.

Since both hard and soft materials can be freely intermixed and the features added during initial fabrication, it is possible to eliminate the difficulties and assembly complexity caused by inserting rigid parts into cavities after the body had been cast as in prior methods.

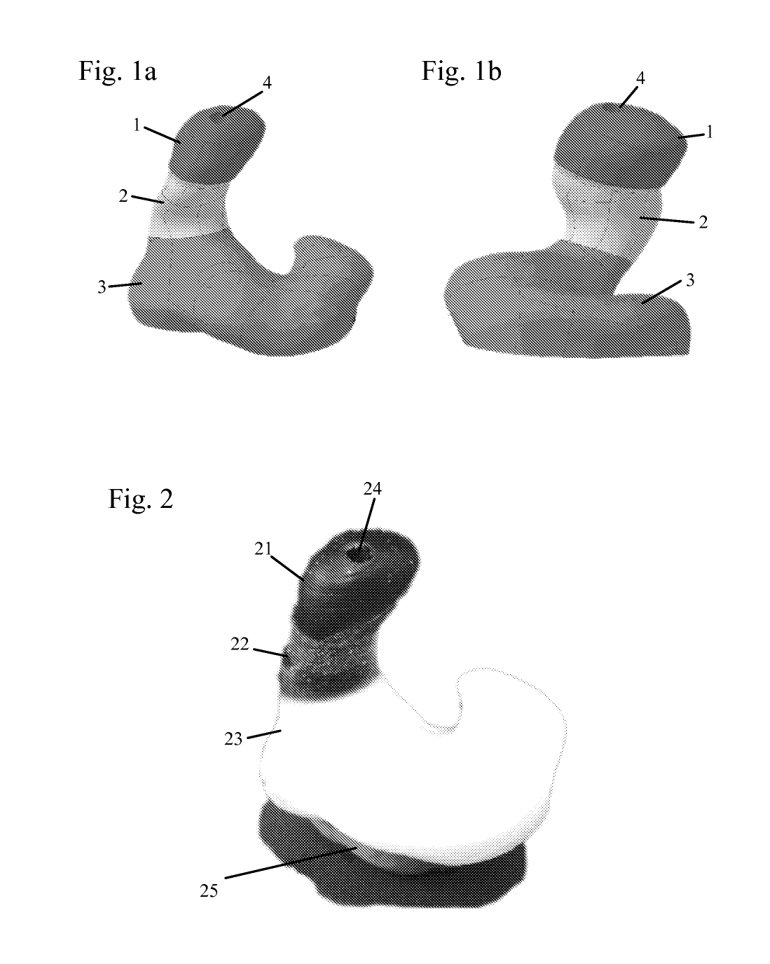

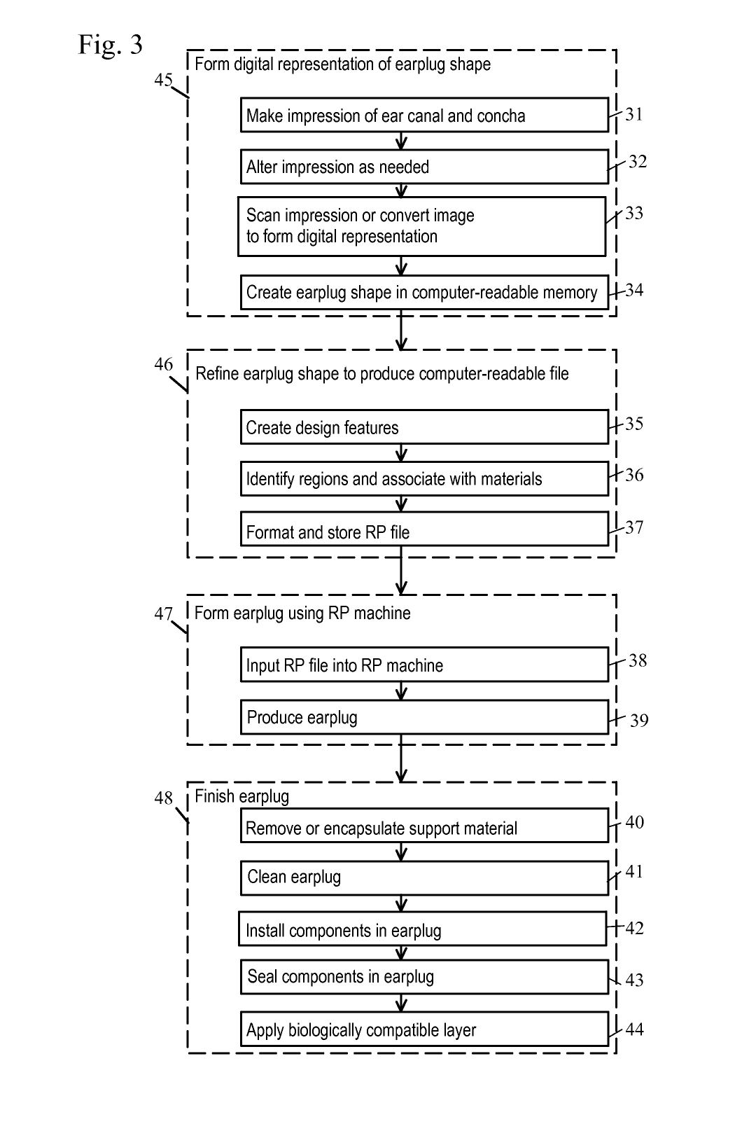

FIGS. 1a and 1b shows two drawings of a custom earplug made with three different material sections. Two views of the same earplug are shown.

Section 1, the section which inserts into the ear canal, is made with a high compliance material. Section 3 sits in the concha region of the ear and is made of hard material. The intermediate section 2 is made with medium compliance material.

The earplug may extend deep into the ear canal, just past the second ear canal bend, so that section 1 is in what is considered the “bony” region and entrance to the bony region. Earplugs inserted into this region achieve the highest noise attenuation; however, the bony region is very sensitive. The material used in section 1 is of high compliance to achieve the greatest comfort. A compliant material with damping characteristics is preferable to a material without damping because the damping reduces mechanical resonance and noise transmission into the unoccluded canal region. The damping property of section 1 can be increased by imbedding the material with material typically used for support in the ink-jet printing process. The material is somewhat waxy and improves damping.

If we use stiffer materials, insertion of the devices is easier. The very soft elastomers used in the multi-material RP process are best described as “lazy” elastomers, which are not particularly springy and have better sound attenuation than the cast silicones or hard materials in use today. This characteristic of the soft RP material gives better performance and retains the advantages of firmness for easier insertion, and flexibility for comfort while changing ear canal shape with jaw motion.

To correct the deficiencies of available materials usable in a multi-material RP machine, a thin biologically compatible compliant layer is applied to finished devices by dipping or spraying after the device has been completed and cleaned of support material. The compliant material chosen, such as silicone, provide lubricity, biocompatibility, and ease of cleaning. Since the surface texture and mechanics may be tailored at miniature dimensions during the RP process, the surfaces to be coated are built to maximize the adherence and reliability of the coatings. If mechanical features are needed to “anchor” the coating in critical spots, they may be designed into the shape of the RP device and will be present in the finished part. Having solved the biocompatibility problem, a range of applications and improvements to conventional methods are enabled by use of multi-material RP technology.

The hole 4 at the distal end of section 1 is used in communications earplugs and hearing aids or other such devices. The hole 4 is sometimes used as a vent, in much smaller diameter, for earplugs to prevent pressurization when inserting and vacuum when removing the earplug. The vent diminishes the low-frequency attenuation of the earplug, but often this is not a problem because low-frequency noise is typically less damaging than high frequency noise (for the same sound pressure level). The distal end of section 1 would not use a vent or sound hole if maximum attenuation is desired.

In section 2, a stiffer, but still compliant material is used. The compliance of the material enables the plug to bend around the canal's first bend when inserting it into the ear canal. However, if this material is too soft, it becomes very difficult to insert the earplug. Section 2 should cover the region near and around the ear canal first bend and up to the ear canal second bend.

A stiffer material, such as hard plastic, is used in section 3. Hard materials are comfortable in the concha region of the ear as evidenced by the wide spread use of hard plastic in-the-ear hearing aids. The hard material facilitates the installation of transducers (such as speakers, microphones, and telecoils) as well as electronics if needed. The stiff material also makes it easier to insert the earplug because the plug will not flex at the base. In addition, if a circuit board is mounted within section 3, bending in this region could damage it.

Shock isolation features can be built into the RP of the shell of the device using the multi-material capability of the process. If this is done, the need for assembly steps and adhesives to add shock mounting and isolation is removed providing higher performance at lower cost.

Since elastomers may be incorporated and bonded to hard shell materials as an integral part of the shell manufacturing process, no assembly labor or additional parts are needed to perform this function. The shape of the seals may be customized on a device-by-device basis, which is not possible in conventional manufacture where seals must be mass produced in moulds. Since the elastomers are bonded to the shell as part of the manufacture, failure rates in the seals are reduced, as is the number of parts making up an assembly thus further reducing assembly and device costs.

The use of multi-material RP permits the entire assembly to be created in one step. Further, since the multi-material RP process permits mixing of hard and soft materials on a micro-droplet level, the process can produce engineered materials with graded hardness to match the requirements of different portions of the ear canal, and the requirements of the internal electronics if desired.

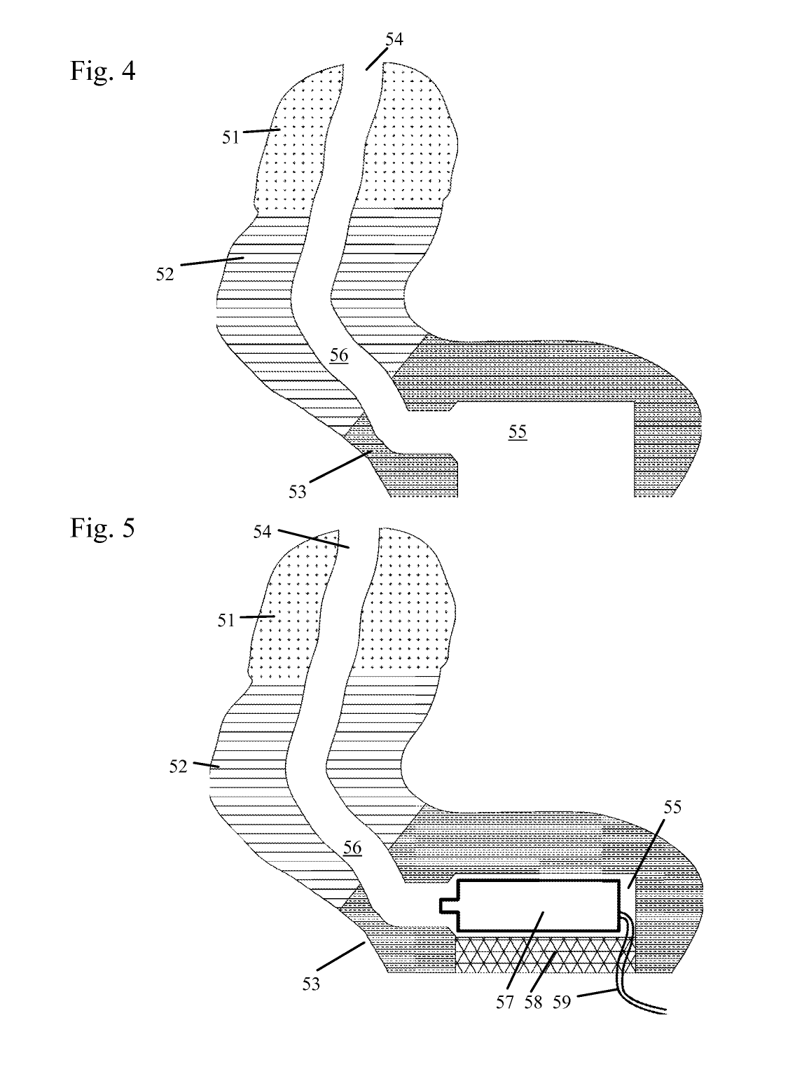

In an RP process, internal passages (as in channel 56 in FIGS. 4 and 5) can be made while the device is being built up, and are limited only by the accuracy and resolution of the process.

Another feature of the multi-material RP systems is their use of a “support material”. This material is an intrinsic part of the process and is a waxy soft substance that is applied by one of the jets in the multi-material head for the purpose of providing a substrate upon which to build up other materials. Usually, this material is washed or dissolved away after the part is completed. The material is non-elastic, and somewhat easy to crumble, which makes it a good damping material. By creating internal cavities in the ear plug and then filling them with support material which is not removed, features may be created to provide improved attenuation.

While the earplug is described above in terms of a three-section earplug, it will be understood that the earplug could be constructed using two separate materials and still maintain a strong advantage over single-material earplugs. In this case, sections 1 and 2 would be made of the same compliant material whereas section 3 would be made of a stiffer material such as hard plastic. If desired, more than three materials could also be used within the teachings of the invention.

FIG. 2 shows a photograph of a physical custom earplug, designed by the inventors, made of three separate material sections using ink-jet printing technology. Section 21 is made with a high compliance material (durometer of 20). Section 22 is made with medium compliance material (durometer of 60), while section 23 is made of hard plastic. (Note that there is clay 25 on the bottom of the plug for photographing purposes.)

The photograph in FIG. 2 was taken before applying an overall coating so that the different sections are visible. A coating is needed if the materials used aren't strictly biocompatible. A sound hole 24 can be seen at the distal end of section 21 so that acoustic communications signals, from a speaker located in section 23, can be delivered to the ear canal.

FIGS. 4 and 5 show sectional views of an earplug. The first section 51 is made of a soft durometer material, the second section 53 is made of a hard durometer material, and the intermediate section 52 has a medium durometer material. A cavity 55 is formed in the second section 53, and a sound channel 56 leads from the cavity 55 to the ear hole 54 in the soft first section 51. A speaker or transducer 57 is inserted into the cavity 55, with its wires 59 leading outward. The cavity 55 with the speaker 57 is sealed by potting material 58 or glue or other material, or a faceplate could be provided instead.

### Method of Manufacturing the Earplug

The method of making the earplug is as follows:
45. First, a digital representation of an earplug shape is formed in computer-readable memory by:
   31. As in the prior art, an impression of the ear canal and concha are made. This can be done by injecting a material into the ear canal, allowing it to harden, and then withdrawing it from the ear to produce an accurate representation of the ear canal shape. Alternative methods, such as medical imaging or photography may be used to create an impression.
   32. If necessary, the impression may be mechanically altered as needed.
   33. The impression is optically or mechanically scanned to produce a digital representation of the ear canal shape, and the digital representation is stored in a computer-readable memory. If medical imaging or photographic techniques are employed, the digital representation is created directly.
   34. This stored representation can be used to create the shape of the earplug in the computer-readable memory, or, if necessary, the digital representation can be further processed and/or modified using a computer program to create the earplug shape, through processes such as trimming and smoothing, in the computer-readable memory.
46. Then, the earplug shape in the computer-readable memory is refined, as needed, to produce a computer-readable file by:
   35. Any required design features, such as a vent, sound delivery tube, cavity for a speaker and/or electronics, are created in the stored representation.
   36. Regions of the earplug design in the computer file are identified and associated with particular materials that will be used in the fabrication process.
   37. The stored representation from step 35 and the region identification and association from step 36 are formatted and stored as a computer RP file in the format required by the rapid prototyping (RP) machine to be used to make the earplug, describing the physical size, geometry, material description, earplug build orientation, build support structures and other parameters of the earplug.
47. Then, the computer RP file of the earplug shape in the computer-readable memory, is used to form the multi-material earplug using a rapid prototyping (RP) machine, by:
   38. The computer RP file from step 37 is inputted into the RP machine.
   39. The RP machine produces the physical earplug.

The RP machine uses nozzles similar to the ones used in inkjet printers, except instead of ink, the nozzles deposit resin materials that can be cured using light. The nozzles are very small and can deposit minute amounts of resin so that a high resolution fabrication can be achieved.

The nozzles deposit “support material” and “part material” resins of varying durometer. The machine deposits support material because the earplug cannot be fabricated suspended in space. The support material fills in voids in the part so that part resin can be deposited. The support material does not bind to the part material, and therefore, can be easily removed once the part has been fabricated. The support material can also be embedded within the part if desired. Multiple nozzles can be used to deposit different resins to achieve a part with multiple mechanical characteristics. In addition, multiple resins can be mixed to achieve a spectrum of durometers.

Once the resin material has been deposited, it is cured with light to maintain its desired shape. Once a build layer is complete, the build platform is moved to make room for the next layer. Part material resins from each layer are bonded together through the light-curing process. This continues until the complete part has been fabricated.
48. The earplug is finished by:
   40. The support material is removed by dissolving, washing, or other compatible process. Some support material may be encapsulated within the earplug, for instance in region 1, to achieve mechanical damping.
   41. The earplug is cleaned using a solvent or soap or other cleaner.
   42. Speakers, microphones, circuit boards and/or other parts are installed into the earplug if desired.
   43. The installed parts are sealed in the earplug using a faceplate, potting material, glue or other method.
   44. A thin biologically compatible silicone layer is applied by dipping or spraying, if desired.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. An earplug comprising a body formed of a plurality of sections made of materials having different hardnesses, in which the plurality of sections are made using a multi-material rapid prototyping system.

2. The earplug of claim 1, in which the plurality of sections of the body comprises:
a first section for insertion into an ear canal, made of a high compliance material; and
a second section for location in a concha region of an ear, made of a hard material.

3. The earplug of claim 2, in which the plurality of sections further comprises an intermediate section between the first section and the second section, made of a material intermediate in hardness between the high compliance material of the first section and the hard material of the second section.

4. The earplug of claim 1, in which the body further comprises a biologically compatible compliant layer covering the body.

5. The earplug of claim 4, in which the biologically compatible compliant layer is made of silicone.

6. The earplug of claim 2, in which the high compliance material of the first section comprises a material with damping characteristics.

7. The earplug of claim 6, in which the material is a soft elastomer imbedded with support material from the multi-material rapid prototyping system.

8. The earplug of claim 3, in which at least part of an interior of the second section is hollow, and the earplug has a passage from the hollow interior of the second section through the intermediate section and the first section, for leading sound into an ear.

9. The earplug of claim 8, further comprising a transducer in the hollow interior part of the second section.

10. A method of making a multi-material earplug having a body formed of a plurality of materials having different hardnesses, the method comprising:
a) creating a digital representation of a shape for the body of the earplug in computer readable memory;
b) refining the digital representation by identifying materials to be used and associating the materials with regions of the shape in the digital representation, resulting in a computer file;
c) inputting the computer file into a multi-material rapid prototyping machine;
d) operating the multi-material rapid prototyping machine to produce the multi-material earplug.

11. The method of claim 10, in which the digital representation of the shape of the earplug is created from an impression of an ear canal and concha of a user.

12. The method of claim 11, in which the impression is made by injecting a material into the ear canal, allowing the material to harden, and then withdrawing the material from the ear to produce an accurate representation of the shape of the ear canal.

13. The method of claim 12, further comprising optically or mechanically scanning the impression to produce the digital representation, and storing the digital representation in computer-readable memory.

14. The method of claim 11 in which the impression is created by taking a digital image of the ear canal and concha, and the digital representation is produced by processing the digital image to produce the digital representation.

15. The method of claim 10, in which the step of refining the digital representation comprises creating design features in the digital representation.

16. The method of claim 15, in which the design features comprises at least one of a vent, sound delivery tube, and a cavity for a speaker or electronics.

17. The method of claim 10, in which the step of refining the digital representation comprises identifying regions of the earplug and associating the regions with materials.

18. The method of claim 10, further comprising removing support material by dissolving, washing, or other compatible process.

19. The method of claim 10, further comprising installing at least one of a speaker, a microphone, or a circuit boards into the earplug.

20. The method of claim 19, further comprising sealing the earplug.

21. The method of claim 20, in which the earplug is sealed using at least one of a faceplate, a potting material, or glue.

22. The method of claim 10, further comprising applying a thin biologically compatible silicone layer to the earplug.
